(19) United States
(12) Patent Application Publication
(10) Pub. No.: US 2012/0237550 A1
(43) Pub. Date: Sep. 20, 2012

(54) Maintaining Antibody-Binding Activity Of Immunosuppressant Drug Conjugates

(75) Inventors: Tie Q. Wei, Wilmington, DE (US); Alan R. Craig, Wilmington, DE (US); Fiore M. Celano, III, Wilmington, DE (US); Samantha Janas, Bear, DE (US)

(73) Assignee: Siemens Healthcare Diagnostics Inc., Deerfield, IL (US)

(21) Appl. No.: 13/049,617

(22) Filed: Mar. 16, 2011

(51) Int. Cl. A61K 38/13; A61P 39/04; G01N 33/53; A61P 37/06; A61K 31/4433; A61K 31/197

(52) U.S. Cl. 424/278.1; 436/501

(57) ABSTRACT

Methods and reagents are disclosed for maintaining the antibody-binding activity of a conjugate of an immunosuppressant drug and a conjugative moiety. The method comprises combining with the conjugate an effective amount of a chelating agent. Compositions include in an aqueous medium (i) a conjugate of an immunosuppressant drug and a conjugative moiety and (ii) a chelating agent in an amount effective to maintain an antibody-binding activity of the conjugate of the immunosuppressant drug and the conjugative moiety. The compositions may be employed in assays for the determination of analytes that include immunosuppressant drug analytes and antibodies for an immunosuppressant drug.

### BACKGROUND

The invention relates to compounds, methods and kits for the determination of immunosuppressant drugs in samples, such as patient samples, known or suspected to contain one or more of such immunosuppressant drugs. In some examples the invention relates more particularly to maintaining the antibody-binding activity of conjugates of immunosuppressant drugs.

The body relies upon a complex immune response system to distinguish self from non-self. At times, the body's immune system must be controlled in order to either augment a deficient response or suppress an excessive response. For example, when organs such as kidney, heart, heart-lung, bone marrow and liver are transplanted in humans, the body will often reject the transplanted tissue by a process referred to as allograft rejection.

In treating allograft rejection, the immune system is frequently suppressed in a controlled manner with drug therapy. Immunosuppressant drugs are carefully administered to transplant recipients in order to help prevent allograft rejection of non-self tissue. Two most commonly administered immunosuppressive drugs to prevent organ rejection in transplant patients are cyclosporine (CSA) and FK-506 (FK or tacrolimus). Another drug that finds use as an immunosuppressant in the United States and other countries is sirolimus, also known as rapamycin. Derivatives of sirolimus are also said to be useful as immunosuppressants. Such derivatives include, for example, everolimus, and the like.

The side effects associated with some immunosuppressant drugs can be controlled in part by carefully controlling the level of the drug present in a patient. Therapeutic monitoring of concentrations of immunosuppressant drugs and related drugs in blood is required to optimize dosing regimes to ensure maximal immunosuppression with minimal toxicity. Although immunosuppressant drugs are highly effective immunosuppressive agents, their use must be carefully managed because the effective dose range is often narrow and excessive dosage can result in serious side effects. On the other hand, too little dosage of an immunosuppressant can lead to tissue rejection. Because the distribution and metabolism of an immunosuppressant drug can vary greatly between patients and because of the wide range and severity of adverse reactions, accurate monitoring of the drug level is essential.

In immunosuppressant drug monitoring, some of the reagents employed are conjugates comprising the immunosuppressant drug chemically linked to another moiety, which may be, for example, a particulate solid support, a label moiety or a binding ligand that binds to a receptor that is conjugated to a solid support. It is important that the activity of such reagents remains relatively constant. Otherwise, errors are caused in assays for the amount of immunosuppressant drug in a sample because the loss of antibody-binding activity of the immunosuppressant drug conjugates reduces the correlation between the amount of the conjugate and the amount of the immunosuppressant drug in a sample. However, reagents comprising an immunosuppressant drug conjugated to a conjugative moiety exhibit reduction in activity due to constituents in a particular sample that is to be analyzed such as, for example, when a sample to be analyzed is whole blood as is often the case in many assays for immunosuppressant drugs.

There is, therefore, a continuing need to develop fast and accurate diagnostic methods to measure levels of immunosuppressant drugs or derivatives thereof in patients. The methods should be capable of being fully automated and be accurate and immunosuppressant drug conjugates should maintain their antibody-binding activity.

### SUMMARY

Some examples in accordance with the principles described herein are directed to methods of maintaining the antibody-binding activity of a conjugate of an immunosuppressant drug and a conjugative moiety. The method comprises combining with the conjugate an effective amount of a chelating agent.

Some examples in accordance with the principles described herein are directed to compositions comprising in an aqueous medium (i) a conjugate of an immunosuppressant drug and a conjugative moiety and (ii) a chelating agent in an amount effective to maintain an antibody-binding activity of the conjugate of the immunosuppressant drug and the conjugative moiety.

Some examples in accordance with the principles described herein are directed to methods for detecting an immunosuppressant drug in a sample. In the method a combination is provided in an assay medium. The combination comprises a sample suspected of containing an immunosuppressant drug, an antibody for the immunosuppressant drug and the aforementioned composition wherein the conjugative moiety is a particle. The combination is examined for the formation of a complex between the antibody for the immunosuppressant drug and the immunosuppressant drug of the sample or for the formation of a complex between the antibody for the immunosuppressant drug and the immunosuppressant drug of the conjugate. The presence of the complex is related to one or both of a presence and an amount of the immunosuppressant drug in the sample.

Some examples in accordance with the present disclosure are directed to methods for detecting an immunosuppressant drug in a sample. A combination is provided in an assay medium, which comprises a sample suspected of containing an immunosuppressant drug, an antibody for the immunosuppressant drug and the aforementioned composition wherein the conjugative moiety is a label. The combination is examined for the formation of a complex between the antibody for the immunosuppressant drug and the immunosuppressant drug. The presence of the complex is related to one or both of a presence and an amount of the immunosuppressant drug in the sample.

Some examples in accordance with the principles described herein are directed to compositions comprising in an aqueous medium (i) a conjugate of sirolimus and a conjugative moiety and (ii) an ethylene diamine tetraacetic acid reagent in an amount effective to maintain an antibody-binding activity of the conjugate of sirolimus and the conjugative moiety.

### DETAILED DESCRIPTION

### General Discussion

Some examples in accordance with the principles described herein are directed to methods of maintaining the antibody-binding activity of a conjugate of an immunosuppressant drug and a conjugative moiety. The method comprises combining the conjugate and an effective amount of a chelating agent.

The current methods focus on the mitigation of inaccurate assay results caused by loss in activity of immunosuppressant drug conjugates employed in an assay. Some examples of methods in accordance with the principles described herein have application, for example, to fully automated homogenous assays in which, prior to the assay, there is no extraction or separation of the immunosuppressant drug from other constituents of the sample including entities in the sample that may have a detrimental effect on the antibody-binding activity of immunosuppressant drug conjugates. In a “non-manual extraction” assay, a sample such as a whole blood sample is combined with a hemolyzing agent and, optionally, a releasing agent in a medium and, following an incubation period to allow for hemolysis and release of the drug from other blood constituents, reagents for conducting an assay for the immunosuppressant drug are added to the medium and the assay is conducted. It has been found that the activity of the immunosuppressant drug conjugates may be compromised for assays in which whole blood samples, for example, are utilized.

In some examples, the present methods are directed to maintaining the antibody-binding activity of a conjugate of an immunosuppressant drug and a conjugative moiety. The phrase “maintaining the antibody-binding activity” refers to maintaining the activity of the conjugate for binding to an antibody for the immunosuppressant drug. In accordance with the principles described herein, the antibody-binding activity of the immunosuppressant drug in the conjugate is substantially maintained. In some examples in accordance with the principles described herein, at least 70% of the antibody-binding activity is maintained, or at least 75% of the antibody-binding activity is maintained, or at least 80% of the antibody-binding activity is maintained, or at least 85% of the antibody-binding activity is maintained, or at least 90% of the antibody-binding activity is maintained, or at least 95% of the antibody-binding activity is maintained, or at least 99% of the antibody-binding activity is maintained, for example.

Immunosuppressant drugs are therapeutic drugs that are administered to transplant recipients in order to help prevent allograft rejection of non-self tissue. Immunosuppressive drugs can be classified into four groups: glucocorticoids, cytostatics, antibodies, drugs acting on immunophilins, and other drugs such as interferons, opiates INF binding proteins, mycophenolate, FTY720 and the like. A particular class of immunosuppressant drugs comprises those drugs that act on immunophilins. Immunophilins are an example of high-affinity, specific binding proteins having physiological significance. Two distinct families of immunophilins are presently known: cyclophilins and macrophilins, the latter of which specifically bind, for example, tacrolimus or sirolimus. The immunosuppressant drugs that act on immunophilin include, for example, cyclosporin (including cyclosporin A, cyclosporin B, cyclosporin C, cyclosporin D, cyclosporin E, cyclosporin F, cyclosporin G, cyclosporin H, cyclosporin I), tacrolimus (FK506, PROGRAF®), sirolimus (rapamycin, RAPAMUNE®), and everolimus (RAD, CERTICAN®), for example.

Immunosuppressant drug conjugates comprise an immunosuppressant drug and a conjugative moiety bound together, optionally through a linking group, to form a single structure. The binding can be either covalent attachment such as by a direct connection, e.g., a chemical bond between the immunosuppressant drug and the conjugative moiety or between the immunosuppressant drug and the conjugative moiety and a linking group, or non-covalent attachment involving specific binding between complementary specific binding pair (sbp) members that are attached to the immunosuppressant drug and the conjugative moiety of the conjugate.

The conjugative moiety is any entity that may be conjugated to the immunosuppressant drug to form a reagent that is employed in an assay for the detection of the immunosuppressant drug. Conjugative moieties include, by way of illustration and not limitation, supports, members of a signal producing system, members of binding pairs such as, for example, ligands and receptors (e.g., biotin-streptavidin or fluorescein-anti-fluorescein antibodies), and macromolecules that provide anchors to a drug analog, for example.

A support may be comprised of an organic or inorganic, solid or fluid, water insoluble material, which may be transparent or partially transparent. The support may be synthetic or naturally-occurring. The support can have any of a number of shapes, such as particle, including bead, film, membrane, tube, well, strip, rod, planar surfaces such as, e.g., plate, paper, etc., fiber, and the like. Depending on the type of assay, the support may or may not be suspendable in the medium in which it is employed. Examples of suspendable supports are polymeric materials such as latex, lipid bilayers or liposomes, oil droplets, cells and hydrogels, metallic particles, and magnetic particles, for example. Other support compositions include polymers, such as cross-linked polysaccharides including agarose and dextran, for example, cellulose, nitrocellulose, cellulose acetate, polyvinyl alcohol, poly(vinyl chloride), polyacrylamide, polyacrylate, polymethacrylates, polyethylene, polypropylene, poly(4-methylbutene), polystyrene, polymethacrylate, poly(ethylene terephthalate), nylon, and poly(vinyl butyrate), for example, either used by themselves or in conjunction with other materials.

The support may be a particle. The particles may have an average diameter of at least about 0.02 microns and not more than about 100 microns. In some embodiments, the particles have an average diameter from about 0.05 microns to about 20 microns, or from about 0.3 microns to about 10 microns. The particle may be organic or inorganic, swellable or non-swellable, porous or non-porous, preferably of a density approximating water, generally from about 0.7 g/mL to about 1.5 g/mL, and composed of material that can be transparent, partially transparent, or opaque. The particles can be biological materials such as cells and microorganisms, e.g., erythrocytes, leukocytes, lymphocytes, hybridomas, streptococcus, Staphylococcus aureus, E. coli, viruses, and the like. The particles can also be particles comprised of organic and inorganic polymers, liposomes, latex particles, metallic particles, magnetic or non-magnetic particles, phospholipid vesicles, chylomicrons, and lipoproteins, for example. In some examples, the particles are chromium dioxide (chrome) particles or latex particles. In some examples, the particles are readily dispersible in an aqueous medium and can be adsorptive or functionalizable so as to permit conjugation to an immunosuppressant drug, either directly or indirectly through a linking group.

Signal producing systems and labels are discussed in more detail hereinbelow in the discussion of various assay systems to which examples in accordance with the principles described herein may be applied. Briefly, a signal producing system (sps) may have one or more components or members, at least one component or member being the label. The signal producing system generates a signal that relates to the presence of an immunosuppressant drug in a sample. The signal producing system includes all of the reagents required to produce a measurable signal. Other components of the signal producing system may be included in a developer solution and can include substrates, enhancers, activators, chemiluminescent compounds, cofactors, inhibitors, scavengers, metal ions, specific binding substances required for binding of signal generating substances, and the like. Other components of the signal producing system may be coenzymes, substances that react with enzymic products, other enzymes and catalysts, for example. The signal producing system provides a signal detectable by external means, by use of electromagnetic radiation, desirably by visual examination. Exemplary signal-producing systems are described in U.S. Pat. No. 5,508,178 (Rose, et al.) and Ullman, et al., U.S. Pat. No. 5,185,243, columns 11-13, the relevant disclosure of which is incorporated herein by reference.

As mentioned above, the label is a member of a signal producing system. The label is capable of being detected directly or is detectable through a specific binding reaction that produces a detectable signal.

For covalent attachment of the components of a conjugate, that is the immunosuppressant drug and the conjugative moiety, one or more of the components contains a functional group suitable for attachment to one or more of the other components. The functional groups suitable for attaching the components may be carbonyl functionalities, both oxocarbonyl, e.g., aldehyde, and non-oxocarbonyl (including nitrogen and sulfur analogs) e.g., carboxy, amidine, amidate, thiocarboxy and thionocarboxy. Alternative functionalities of oxo include active halogen, diazo, mercapto, olefin, particularly activated olefin, amino, phosphoro and the like. Of particular interest are activated esters or alkylating agents. Details of techniques for attaching molecules to one another may be found, for example, in Matthews, et al., Anal. Biochem. (1985) 151:205-209; Engelhardt, et al., European Patent Application No. 0302175 and U.S. Pat. No. 3,817,837, the relevant disclosure of which is incorporated herein by reference in its entirety.

As indicated above, the components, i.e., immunosuppressant drug and conjugative moiety, of the reagents may be attached together non-covalently. For example, a small organic molecule such as, but not limited to, biotin including bis-biotin, and fluorescein, for example, may be incorporated into one of the components and the other component may be linked to a binding partner for the small organic molecule such as, for example, respectively, streptavidin and anti-fluorescein. The binding of the binding partners results in the non-covalent attachment of the components to one another.

As mentioned above, the method comprises combining the conjugate and an effective amount of a chelating agent. In some examples in accordance with the principles described herein, the chelating agent may be, but is not limited to, ethylenediamine-tetraacetic acid (EDTA), trans-1,2-diamino-cyclohexan-N,N,N′,N′-tetraacetic acid (CDTA), ethylene glycol-O,O′-bis-(2-aminoethyl)-N,N,N′,N′-tetraacetic acid (EGTA), diethylenetriamine-pentaacetic acid (DTPA), N-(2-hydroxyethyl)-ethylenediamine-N,N′,N′-triacetic acid (HEDTA), nitrilotriacetic acid (NTA), nitrilo-2,2′,2″-triacetic acid, diethylenetriamine-N,N,N′,N′,N″-pentaacetic acid, triethylenetetramin-N,N,N′,N″,N′″,N′″-hexaacetic acid (TTHA), methylamine, histidine, malate and phytochelatin, hemoglobin, chlorophyll, siderophore, pyocyanin, pyoverdin, Enterobactin, peptides and sugars, humic acid, citric acid, water softeners, phosphonates, tetracycline, gadolinium, organophosphorus compound 2,2′-bis(diphenylphosphino)-1,1′-binaphthyl, Pentetic acid; N,N-Bis(2-(bis-(carboxymethyl)amino)ethyl)-glycine, (N,N-bis(carboxymethyl)glycine, triglycollamic acid; [[(Carboxymethyl)imino]bis-(ethylenenitrilo)]-tetraacetic acid), Trilone A, α,α′,α″-trimethylaminetricarboxylic acid, tri(carboxymethyl)amine, aminotriacetic acid, Titriplex i, and Hampshire NTA acid, for example, and appropriate salts of any of the above.

The chelating agent is present in the combination in an amount that is effective to maintain the antibody-binding activity of the immunosuppressant drug of the conjugate. The amount of the chelating agent is dependent on one or more of the nature and amount of the conjugate, the nature of the immunosuppressant drug analyte, and an amount of divalent metal ions that may be leached from metal parts of processing equipment such as, e.g., equipment for forming tablets, for example. In some examples in accordance with the principles described herein, an effective amount of the chelating agent in the combination may be, for example, about 0.1% to about 10%, or about 0.5% to about 10%, or about 1% to about 10%, or about 2% to about 10%, or about 5% to about 10%, or about 7% to about 10%, or about 0.1% to about 8%, or about 0.5% to about 8%, or about 1% to about 8%, or about 2% to about 8%, or about 5% to about 8%, or about 7% to about 8%, or about 0.1% to about 6%, or about 0.5% to about 6%, or about 1% to about 6%, or about 2% to about 6%, or about 5% to about 6%, or about 0.1% to about 5%, or about 0.5% to about 5%, or about 1% to about 5%, or about 2% to about 5%, or about 3% to about 5%, for example. Above percentages are by weight of the combination in a suitable medium.

As mentioned above, some examples in accordance with the principles described herein are directed to compositions comprising in an aqueous medium (i) a conjugate of an immunosuppressant drug and a conjugative moiety and (ii) a chelating agent in an amount effective to maintain an antibody-binding activity of the conjugate of the immunosuppressant drug and the conjugative moiety. The aqueous medium may be solely water or may include about 0.1% to about 80%, or 0.1% to about 60%, or about 0.1 to about 40%, or about 0.1% to about 30%, or about 0.1% to about 20%, or about 0.1% to about 10%, or about 0.1% to about 5%, or about 1% to about 80%, or 1% to about 60%, or about 1% to about 40%, or about 1% to about 30%, or about 1% to about 20%, or about 1% to about 10%, or about 1% to about 5%, or about 5% to about 80%, or 5% to about 60%, or about 5% to about 40%, or about 5% to about 30%, or about 5% to about 20%, or about 5% to about 10%, for example, of a cosolvent. Above percentages are by volume of the medium. The cosolvent may be, for example, but not limited to, an organic solvent such as, for example, an alcohol, an ester, an ether, an amide or an amine. Particular examples of co-solvents include, by way of illustration and not limitation, polyols comprising about 2 to about 6 carbon atoms, 2 to 3 hydroxy groups (for example, ethylene glycol, propylene glycol, and glycerol), sulfoxides comprising about 2 to about 6 carbon atoms (for example, dimethyl sulfoxide and diethyl sulfoxide), sulfones comprising about 2 to about 6 carbon atoms (for example, dimethyl sulfone, diethyl sulfone and sulfolane), amides comprising about 2 to about 6 carbon atoms (for example, formamides (e.g., dimethyl formamide and diethyl formamide), N-methyl pyrrolidone, tetramethyl urea, and dimethylacetamide), mono-, di- and tri-ethers of a polyol comprising about 2 to about 6 carbon atoms and 2 to 3 hydroxy groups (for example, 1-methoxy-2-propanol and 1,2-dimethoxy propanol), and mono-, di- and tri-esters of a polyol comprising about 2 to about 6 carbon atoms and 2 to 3 hydroxy groups (for example, 2-hydroxypropyl acetate and bis(2-methoxyethyl)ether(diglyme)), for example. Two or more of the above co-solvents may be employed in combination.

The pH for the medium will usually be in the range of about 4 to about 11, or in the range of about 5 to about 10, or in the range of about 6.5 to about 9.5, for example. Various buffers may be used to achieve the desired pH and maintain the pH of the medium. Illustrative buffers include borate, phosphate, carbonate, tris, and barbital, for example. The particular buffer employed is not critical, but in an individual composition one or another buffer may be preferred. Various ancillary materials also may be included in the medium. For example, in addition to buffers the medium may comprise stabilizers for the medium or other components of the medium, preservatives for long term shelf life, non-specific binding blockers to prevent false results, and detergents, e.g., to keep a hydrophobic drug from binding to a plastic container.

The compositions in accordance with the principles described herein may be employed in methods of detecting an immunosuppressant drug or an antibody for an immunosuppressant drug in a sample. The sample to be analyzed is one that is suspected of containing one or more immunosuppressant drug analytes. The samples are preferably from humans or animals and include, but are not limited to, biological fluids such as whole blood, serum, plasma, sputum, lymphatic fluid, semen, vaginal mucus, feces, urine, spinal fluid, saliva, stool, cerebral spinal fluid, tears, and mucus, for example, and biological tissue such as hair, skin, sections or excised tissues from organs or other body parts, for example. In many instances, the sample is whole blood, plasma or serum and, in a particular example, the sample is whole blood. The sample may or may not be pretreated to remove endogenous binding moieties that bind to the immunosuppressant drug.

The sample can be prepared in any convenient medium that does not interfere with an assay; an aqueous medium generally is employed. The nature of the medium is discussed in more detail below. Depending on the nature of the sample, one or more pretreatments may be carried out on the sample such as, by way of illustration and not limitation, pretreatment with a hemolytic agent, pretreatment with a releasing agent, pretreatment with a drug displacer that displaces bound drug from its endogenous binding partners such as proteins, and hemolytic detergent(s) that assist in lysing blood cells to release drug from the blood cells, for example. One or more of the pretreatment agents may be combined in a medium, which is usually an aqueous medium and is referred to herein as a pretreatment medium. All of the pretreatment agents may be combined simultaneously in the medium or one or more of the pretreatment reagents may be added sequentially. The medium may also comprise one or more preservatives as are known in the art such as, for example, sodium azide, neomycin sulfate, PROCLIN® 300, Streptomycin, and the like. The pH for the pretreatment medium may be in the range of about 4 to about 11, or in the range of about 5 to about 10, or in the range of about 6.5 to about 9.5, for example. The pretreatment agents are present in a concentration or amount sufficient to achieve the desired effect or function, such as, for example, hemolysis or release of immunosuppressant drug from endogenous binding substances. The medium is then incubated under conditions and for a time to achieve the desired function for the various pretreatment agents.

Following the above incubation period, reagents for determining one or both of the presence and amount of the immunosuppressant drug in the sample are added to the medium. The nature of the reagents is dependent on the particular type of assay to be performed. In general, the assay is a method for the determination or measuring of one or both of the presence and amount of an immunosuppressant drug analyte. Various assay methods are discussed below by way of illustration and not limitation.

In many embodiments the reagents comprise at least one antibody for the immunosuppressant drug. By the phrase “antibody for the immunosuppressant drug” is meant an antibody that binds specifically to the immunosuppressant drug and does not bind to any significant degree to other substances that would distort the analysis for the immunosuppressant drug.

Antibodies specific for an immunosuppressant drug for use in immunoassays can be monoclonal or polyclonal. Such antibodies can be prepared by techniques that are well known in the art such as immunization of a host and collection of sera (polyclonal) or by preparing continuous hybrid cell lines and collecting the secreted protein (monoclonal) or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, and IgM, for example. Fragments thereof may include Fab, Fv and F(ab')₂, and Fab', for example. In addition, aggregates, polymers, and conjugates of immunoglobulins or their fragments can be used where appropriate so long as binding affinity for a particular molecule is maintained.

As discussed above, an antibody selected for use in an immunoassay for an immunosuppressant drug, for example, should specifically and preferentially bind the immunosuppressant drug and its pharmaceutically active metabolites over other ligands such as other metabolites or related drugs. For example, an antibody for tacrolimus should specifically and preferentially bind tacrolimus over, e.g., rapamycin, and vice versa. In general, an antibody should be capable of distinguishing between one immunosuppressant drug relative to a second immunosuppressant drug.

Other reagents are included in the assay medium depending on the nature of the assay to be conducted. Such assays usually involve reactions between binding partners such as an immunosuppressant drug analyte and a corresponding antibody or the binding between an antibody and a corresponding binding partner such as, for example, a second antibody that binds to the first antibody. Accordingly, the binding partner may be a protein, which may be an antibody or an antigen. The binding partner may be a member of a specific binding pair (“sbp member”), which is one of two different molecules, having an area on the surface or in a cavity, which specifically binds to and is thereby defined as complementary with a particular spatial and polar organization of the other molecule. The members of the specific binding pair will usually be members of an immunological pair such as antigen-antibody, although other specific binding pairs such as biotin-avidin, hormones-hormone receptors, enzyme-substrate, nucleic acid duplexes, IgG-protein A, polynucleotide pairs such as DNA-DNA, DNA-RNA, for example, are not immunological pairs but are included within the scope of sbp member.

As discussed above, specific binding involves the specific recognition of one of two different molecules for the other compared to substantially less recognition of other molecules. On the other hand, non-specific binding involves non-covalent binding between molecules that is relatively independent of specific surface structures. Non-specific binding may result from several factors including hydrophobic interactions between molecules. Preferred binding partners are antibodies.

The present disclosure has application to many types of immunoassays that may be employed to determine one or both of the presence and amount of analytes that include, for example, an immunosuppressant drug analyte, or an antibody therefor, in a sample suspected of containing such analyte. The immunoassays may involve labeled or non-labeled reagents. Immunoassays involving non-labeled reagents usually comprise the formation of relatively large complexes involving one or more antibodies. Such assays include, for example, immunoprecipitin and agglutination methods and corresponding light scattering techniques such as, e.g., nephelometry and turbidimetry, for the detection of antibody complexes. For the non-labeled assays, a composition in accordance with the principles described herein may be employed where the composition comprises a conjugate of the immunosuppressant drug and a support such as, for example, a particle. The composition also comprises a chelating agent in an amount effective to maintain an antibody-binding activity of the immunosuppressant drug-support conjugate. In one example, the immunosuppressant drug analyte in a sample competes with the immunosuppressant drug conjugate such that the more the amount of immunosuppressant drug in a sample, the less will be the amount of a precipitate formed by agglutination. In some examples the analyte is an antibody for the immunosuppressant drug that may be present in a sample and a conjugate in accordance with the principles described herein having an immunosuppressant drug bound to a particulate support is employed. The presence of the antibody in a sample results in the agglutination of the conjugate reagent.

Labeled immunoassays include enzyme immunoassays, fluorescence polarization immunoassays, radioimmunoassay, inhibition assays, induced luminescence, fluorescent oxygen channeling assay, and so forth. In the labeled assay approach, an immunosuppressant conjugate having an immunosuppressant drug bound to a label such as, for example, an enzyme, can compete with immunosuppressant drug in a sample such that the greater the amount of immunosuppressant drug in the sample, the less will be the amount of signal from the label.

As mentioned above, in many of the assays discussed herein, a label is employed and is in many examples part of the immunosuppressant drug conjugate. On the other hand that label may be part of a reagent independent of the immunosuppressant drug conjugate. The label is usually part of a signal producing system (“sps”). The nature of the label is dependent on the particular assay format. A signal producing system usually includes one or more components, at least one component being a detectable label, which generates a detectable signal that relates to the amount of bound and/or unbound label, i.e. the amount of label bound or not bound to the immunosuppressant drug being detected or to an agent that reflects the amount of the immunosuppressant drug to be detected. The label is any molecule that produces or can be induced to produce a signal, and may be, for example, a fluorescer, radiolabel, enzyme, chemiluminescer or photosensitizer. Thus, the signal is detected and/or measured by detecting enzyme activity, luminescence, light absorbance or radioactivity, and so forth, as the case may be.

In some examples, the labels are radioisotopic, luminescent, particulate or enzymic. The label can be a poly(amino acid), or protein, or non-poly(amino acid), isotopic or non-isotopic, usually non-isotopic, and can be a catalyst, such as an enzyme, a polynucleotide coding for a catalyst, promoter, dye, fluorescent molecule, chemiluminescent molecule, coenzyme, enzyme substrate, radioactive group, a small organic molecule, amplifiable polynucleotide sequence, a particle such as latex or carbon particle, metal sol, crystallite, liposome, cell, etc., which may or may not be further labeled with a dye, catalyst or other detectable group, and the like.

The term “non-poly(amino acid) labels” refers to those labels that are not proteins. A non-poly(amino acid) label may be a member of a signal producing system. The non-poly(amino acid) label is capable of being detected directly or is detectable through a specific binding reaction that produces a detectable signal. The non-poly(amino acid) labels generally are radioisotopic, luminescent (such as, e.g., acridinium esters), particulate (such as, e.g., magnetic particles that can be separated bound from un-bound, latex particles that can be measured by turbidity and nephelometry, and chemiluminescence beads (e.g., LOCI chemibeads), for example. The label can be isotopic or non-isotopic, usually non-isotopic, and can be a polynucleotide coding for a catalyst, promoter, dye, fluorescent molecule, chemiluminescent molecule, coenzyme, enzyme substrate, radioactive group, a small organic molecule, amplifiable polynucleotide sequence, a particle such as latex or carbon particle, metal sol, crystallite, liposome, cell, etc., which may or may not be further labeled with a dye, catalyst or other detectable group, and the like. Poly(amino acid) labels include, by way of illustration and not limitation, peptides and proteins such as e.g., enzymes, for example.

Suitable labels include, by way of illustration and not limitation, enzymes such as alkaline phosphatase, glucose-6-phosphate dehydrogenase (“G6PDH”) and horseradish peroxidase; ribozyme; a substrate for a replicase such as QB replicase; promoters; dyes; fluorescers, such as fluorescein, isothiocyanate, rhodamine compounds, phycoerythrin, phycocyanin, allophycocyanin, o-phthaldehyde, and fluorescamine; complexes such as those prepared from CdSe and ZnS present in semiconductor nanocrystals known as Quantum dots; chemiluminescers such as isoluminol; sensitizers; coenzymes; enzyme substrates; radiolabels such as ¹²⁵1, ¹³¹1, ¹⁴C, ³H, ⁵⁷Co and ⁷⁵Se; particles such as latex particles, carbon particles, metal particles including magnetic particles, e.g., chromium dioxide (CrO₂) particles, and the like; metal sol; crystallite; liposomes; cells, etc., which may be further labeled with a dye, catalyst or other detectable group. Suitable enzymes and coenzymes are disclosed in Litman, et al., U.S. Pat. No. 4,275,149, columns 19-28, and Boguslaski, et al., U.S. Pat. No. 4,318,980, columns 10-14; suitable fluorescers and chemiluminescers are disclosed in Litman, et al., U.S. Pat. No. 4,275,149, at columns 30 and 31; which are incorporated herein by reference.

The label can directly produce a signal and, therefore, additional components are not required to produce a signal. Numerous organic molecules, for example fluorescers, are able to absorb ultraviolet and visible light, where the light absorption transfers energy to these molecules and elevates them to an excited energy state. This absorbed energy is then dissipated by emission of light at a second wavelength. Other labels that directly produce a signal include radioactive isotopes and dyes.

Alternately, the label may need other components to produce a signal, and the signal producing system would then include all the components required to produce a measurable signal. Such other components may include substrates, coenzymes, enhancers, additional enzymes, substances that react with enzymic products, catalysts, activators, cofactors, inhibitors, scavengers, metal ions, and a specific binding substance required for binding of signal generating substances.

Enzymes of particular interest as label proteins are redox enzymes, particularly dehydrogenases such as glucose-6-phosphate dehydrogenase, lactate dehydrogenase, etc., and enzymes that involve the production of hydrogen peroxide and the use of the hydrogen peroxide to oxidize a dye precursor to a dye. Particular combinations include saccharide oxidases, e.g., glucose and galactose oxidase, or heterocyclic oxidases, such as uricase and xanthine oxidase, coupled with an enzyme which employs the hydrogen peroxide to oxidize a dye precursor, that is, a peroxidase such as horse radish peroxidase, lactoperoxidase, or microperoxidase. Additional enzyme combinations are known in the art. When a single enzyme is used as a label, other enzymes may find use such as hydrolases, transferases, and oxidoreductases, preferably hydrolases such as alkaline phosphatase and beta-galactosidase. Alternatively, luciferases may be used such as firefly luciferase and bacterial luciferase. Illustrative co-enzymes that find use include NAD[H], NADP[H], pyridoxal phosphate, FAD[H], FMN[H], etc., usually coenzymes involving cycling reactions. See, for example, U.S. Pat. No. 4,318,980, the disclosure of which is incorporated herein by reference.

With label proteins such as, for example, enzymes, the molecular weight range will be from about 10,000 to about 600,000, or from about 10,000 to about 300,000 molecular weight. There is usually at least about 1 immunosuppressant drug analog per about 200,000 molecular weight, or at least about 1 per about 150,000 molecular weight, or at least about 1 per about 100,000 molecular weight, or at least about 1 per about 50,000 molecular weight, and so forth. In the case of enzymes, the number of immunosuppressant drug analog groups is usually from 1 to about 20, about 2 to about 15, about 3 to about 12, or about 6 to about 10.

One general group of immunoassays that may be employed includes immunoassays using a limited concentration of antibody. Another group of immunoassays involves the use of an excess of one or more of the principal reagents such as, for example, an excess of an antibody for the immunosuppressant drug. Another group of immunoassays are separation-free homogeneous assays in which the labeled reagents modulate the label signal upon immunosuppressant drug-antibody binding reactions. Another group of assays includes labeled antibody reagent limited competitive assays for immunosuppressant drug. In this type of assay, an immunosuppressant drug-support conjugate in accordance with the principles described herein is present in a constant, limited amount. The partition of a label between the immobilized immunosuppressant drug analyte and free immunosuppressant drug analyte depends on the concentration of analyte in the sample.

The assays can be performed either without separation (homogeneous) or with separation (heterogeneous) of any of the assay components or products. Homogeneous immunoassays are exemplified by the EMIT® assay (Syva Company, San Jose, Calif.) disclosed in Rubenstein, et al., U.S. Pat. No. 3,817,837, column 3, line 6 to column 6, line 64; immunofluorescence methods such as those disclosed in Ullman, et al., U.S. Pat. No. 3,996,345, column 17, line 59, to column 23, line 25; enzyme channeling immunoassays (“ECIA”) such as those disclosed in Maggio, et al., U.S. Pat. No. 4,233,402, column 6, line 25 to column 9, line 63; the fluorescence polarization immunoassay (“FPIA”) as disclosed, for example, in, among others, U.S. Pat. No. 5,354,693; and so forth.

Other enzyme immunoassays are the enzyme modulate mediated immunoassay (“EMMIA”) discussed by Ngo and Lenhoff, FEBS Lett. (1980) 116:285-288; the substrate labeled fluorescence immunoassay (“SLFIA”) disclosed by Oellerich, J. Clin. Chem. Clin. Biochem. (1984) 22:895-904; the combined enzyme donor immunoassays (“CEDIA”) disclosed by Khanna, et al., Clin. Chem. Acta (1989) 185:231-240; homogeneous particle labeled immunoassays such as particle enhanced turbidimetric inhibition immunoassays (“PETINIA”), particle enhanced turbidimetric immunoassay (“PETIA”), etc.; and the like.

Other assays include the sol particle immunoassay (“SPIA”), the disperse dye immunoassay (“DIA”); the metalloimmunoassay (“MIA”); the enzyme membrane immunoassays (“EMIA”); luminoimmunoassays (“LIA”); and so forth. Other types of assays include immunosensor assays involving the monitoring of the changes in the optical, acoustic and electrical properties of an antibody-immobilized surface upon the binding of an immunosuppressant drug. Such assays include, for example, optical immunosensor assays, acoustic immunosensor assays, semiconductor immunosensor assays, electrochemical transducer immunosensor assays, potentiometric immunosensor assays, and amperometric electrode assays.

In some embodiments multi-analyte immunoassays may be utilized where the immunosuppressant drug analyte may be the subject of detection along with one or more other analytes such as other drugs and the like. Such multi-analyte systems are described, for example, in Loor, et al., J. Anal. Toxicol. 12: 299 (1988).

The assays discussed above are normally carried out in an aqueous buffered medium at a moderate pH, generally that which provides optimum assay sensitivity. The pH for the assay medium may be in the range of about 4 to about 11, or in the range of about 5 to about 10, or in the range of about 6.5 to about 9.5, for example. The pH will usually be a compromise between optimum binding of the binding members of any specific binding pairs, the pH optimum for other reagents of the assay such as members of the signal producing system, and so forth.

Various buffers may be used to achieve the desired pH and maintain the pH during the determination. Illustrative buffers include borate, phosphate, carbonate, tris, barbital and the like. The particular buffer employed is not critical, but in an individual assay one or another buffer may be preferred. Various ancillary materials may be employed in the above methods. For example, in addition to buffers the medium may comprise stabilizers for the medium and for the reagents employed. Frequently, in addition to these additives, proteins may be included, such as albumins; quaternary ammonium salts; polyanions such as dextran sulfate; binding enhancers, or the like.

One or more incubation periods may be applied to the medium at one or more intervals including any intervals between additions of various reagents mentioned above. The medium is usually incubated at a temperature and for a time sufficient for binding of various components of the reagents to occur. Moderate temperatures are normally employed for carrying out the method and usually constant temperature, preferably, room temperature, during the period of the measurement. Incubation temperatures normally range from about 5° C. to about 99° C., or from about 15° C. to about 70° C., or from about 20° C. to about 45° C., for example. The time period for the incubation is about 0.2 seconds to about 24 hours, or about 1 second to about 6 hours, or about 2 seconds to about 1 hour, or about 1 to about 15 minutes. The time period depends on the temperature of the medium and the rate of binding of the various reagents, which is determined by the association rate constant, the concentration, the binding constant and dissociation rate constant. Temperatures during measurements will generally range from about 10° C. to about 50° C., or from about 15° C. to about 40° C., for example.

The concentration of analyte that may be assayed generally varies from about 10⁻⁵ to about 10⁻¹⁷ M, more usually from about 10⁻⁶ to about 10⁻¹⁴ M. Considerations, such as whether the assay is qualitative, semi-quantitative or quantitative (relative to the amount of immunosuppressant drug analyte present in the sample), the particular detection technique and the concentration of the analyte normally determine the concentrations of the various reagents.

The concentrations of the various reagents in the assay medium will generally be determined by the concentration range of interest of the analyte such as, e.g., an immunosuppressant drug analyte, the nature of the assay, and the like. However, the final concentration of each of the reagents is normally determined empirically to optimize the sensitivity of the assay over the range. That is, a variation in concentration of immunosuppressant drug analyte that is of significance should provide an accurately measurable signal difference. Considerations such as the nature of the signal producing system and the nature of the analytes normally determine the concentrations of the various reagents.

While the order of addition may be varied widely, there will be certain preferences depending on the nature of the assay. The simplest order of addition is to add all the materials simultaneously and determine the effect that the assay medium has on the signal as in a homogeneous assay. Alternatively, the reagents can be combined sequentially. In some examples, an incubation step may be involved subsequent to each addition as discussed above.

### Examination Step

In a next step of the method in accordance with the present disclosure, the medium is examined for the presence of a complex comprising the immunosuppressant drug and the antibody for the immunosuppressant drug. The presence and/or amount of the complex indicates the presence and/or amount of the immunosuppressant drug in the sample.

The phrase “measuring the amount of an immunosuppressant drug analyte” refers to the quantitative, semiquantitative and qualitative determination of the immunosuppressant drug analyte. Methods that are quantitative, semiquantitative and qualitative, as well as all other methods for determining the immunosuppressant drug analyte, are considered to be methods of measuring the amount of the immunosuppressant drug analyte. For example, a method, which merely detects the presence or absence of the immunosuppressant drug analyte in a sample suspected of containing the immunosuppressant drug analyte, is considered to be included within the scope of the present invention. The terms “detecting” and “determining,” as well as other common synonyms for measuring, are contemplated within the scope of the present invention.

In many embodiments the examination of the medium involves detection of a signal from the medium. One or both of the presence and amount of the signal are related to the presence and/or amount of the immunosuppressant drug in the sample. The particular mode of detection depends on the nature of the signal producing system. As discussed above, there are numerous methods by which a label of a signal producing system can produce a signal detectable by external means, desirably by visual examination, and include, for example, electromagnetic radiation, electrochemistry, heat, radioactivity detection, and chemical reagents.

Activation of a signal producing system depends on the nature of the signal producing system members. For those members of a signal producing system that are activated with light, the member is irradiated with light. For members of signal producing systems that are on the surface of a particle, addition of a base may result in activation. Other activation methods will be suggested to those skilled in the art in view of the disclosures herein. For some signal producing systems, no agent for activation is necessary such as those systems that involve a label that is a radioactive label, an enzyme, and so forth. For enzyme systems, addition of a substrate and/or a cofactor may be necessary.

The examination for presence and/or amount of the signal also includes the detection of the signal, which is generally merely a step in which the signal is read. The signal is normally read using an instrument, the nature of which depends on the nature of the signal. The instrument may be a spectrophotometer, fluorometer, absorption spectrometer, luminometer, chemiluminometer, actinometer, photographic instrument, and the like. The presence and amount of signal detected is related to the presence and amount of the immunosuppressant drug compound present in a sample. Temperatures during measurements generally range from about 10° C. to about 70° C., or from about 20° C. to about 45° C., or about 20° C. to about 25° C., for example. In one approach standard curves are formed using known concentrations of the analytes to be screened. As discussed above, calibrators and other controls may also be used.

### Specific Embodiments of Assays

The following examples describe specific examples of the invention by way of illustration and not limitation and are intended merely to describe, and not to limit, the scope of the present disclosure and the appended claims.

In a homogeneous assay after all of the reagents have been combined, the signal is determined and related to the amount of analyte in the sample. For example, in an EMIT® assay for a immunosuppressant drug, a sample suspected of containing the immunosuppressant drug is combined in an aqueous medium either simultaneously or sequentially with an antibody capable of recognizing the immunosuppressant drug, and a reagent that comprises a conjugate of the immunosuppressant drug and an enzyme where the conjugate reagent comprises a chelating agent in accordance with the principles described herein. A substrate for the enzyme is added, which results in the formation of a chromogenic or fluorogenic product upon enzyme-catalyzed reaction. Examples of enzymes are glucose-6-phosphate dehydrogenase and alkaline phosphatase but other enzymes may be employed. The immunosuppressant drug analyte and the immunosuppressant drug moiety of the enzyme conjugate compete for binding sites on the antibody. The enzyme activity in the medium is then determined, usually by spectrophotometric means, and is compared to the enzyme activity determined when calibrators or reference samples are tested, in which a known amount of the immunosuppressant drug is present. Typically, the calibrators are tested in a manner similar to the testing of the sample suspected of containing the immunosuppressant drug analytes. The calibrators contain differing, but known concentrations of the immunosuppressant drug analyte to be determined. In most examples, the concentration ranges present in the calibrators span the range of suspected immunosuppressant drug analyte concentrations in unknown samples.

Heterogeneous assays usually involve one or more separation steps and can be competitive or non-competitive. A variety of competitive and non-competitive assay formats are disclosed in Davalian, et al., U.S. Pat. No. 5,089,390, column 14, line 25 to column 15, line 9, which disclosure is incorporated herein by reference. In one type of competitive assay, a support, as discussed herein, having antibodies for the immunosuppressant drug bound thereto is contacted with a medium containing the sample and a reagent that is a conjugate of the immunosuppressant drug and an enzyme wherein the conjugate reagent comprises a chelating agent in accordance with the principles described herein. After separating the support and the medium, the enzyme activity of the support or the medium is determined by conventional techniques and related to one or both of the presence and amount of the immunosuppressant drug in the sample. In certain examples a second enzyme may be employed in addition to the enzyme of the enzyme conjugate. The enzymes of the pair of enzymes are related in that a product of the first enzyme serves as a substrate for the second enzyme.

Another example of an assay format is a capture assay. In this assay format, the antibody for the immunosuppressant drug is covalently bound to a magnetic particle. The sample is incubated with these particles to allow the immunosuppressant drug in the sample to bind to the antibodies for the immunosuppressant drug. Subsequently, a reagent that comprises a conjugate of the immunosuppressant drug bound to an enzyme, wherein the conjugate reagent comprises a chelating agent in accordance with the principles described herein, is incubated with the magnetic particles. After washing, the amount of enzyme that is bound to the magnetic particles is measured and is inversely related to one or both of the presence and amount of the immunosuppressant drug in the sample.

The following specific assay descriptions are by way of illustration of, and not as a limitation on, the scope of the present invention. Selection of sirolimus as the immunosuppressant drug is also by way of illustration and not limitation since the examples in accordance with the principles described herein have general application to detection of immunosuppressant drugs in general.

In one example in accordance with the present disclosure, a test sample or a sirolimus standard is mixed with a conjugate reagent that is a conjugate of sirolimus and biotin wherein the conjugate reagent comprises a chelating agent in accordance with the principles described herein. The sirolimus of the test sample and the sirolimus of the conjugate reagent are allowed to compete for binding to the antibody for the sirolimus, which is capable of binding to sirolimus analyte or the sirolimus moiety of the conjugate reagent. After rinsing with an appropriate wash buffer, a detection molecule consisting of streptavidin or avidin conjugated to an enzyme, florescent or chemiluminescent molecule or radioactive moiety can be added to the medium, which is then examined for one or both of the presence and amount of signal. The presence and/or amount of signal is related to the presence and/or amount of sirolimus.

In one example in accordance with the principles described herein, the assay employed is an induced luminescence assay, which is described in U.S. Pat. No. 5,340,716 (Ullman, et al.) and which disclosure is incorporated herein by reference. The reagents include two latex bead reagents and a biotinylated anti-sirolimus mouse monoclonal antibody. The first bead reagent is a conjugate wherein one latex bead that contains a chemiluminescent dye is coated with sirolimus. The conjugate reagent comprises a chelating agent in accordance with the principles described herein. The second bead reagent is coated with streptavidin and contains a photosensitizer dye. In a first step, sample suspected of containing sirolimus is incubated with biotinylated antibody for sirolimus, which allows sirolimus from the sample to saturate a fraction of the biotinylated antibody where the fraction is directly related to the sirolimus concentration in the medium. In a second step, the first bead reagent is added and leads to the formation of bead-biotinylated antibody immunocomplexes with the non-saturated fraction of the biotinylated antibody. The second bead reagent is then added and binds to the biotin to form bead pair immunocomplexes. When illuminated by light at 680 nm, the second bead reagent converts dissolved oxygen in the reaction solution into the more energetic singlet oxygen form. In the bead pairs, the singlet oxygen diffuses into the first bead reagent thereby triggering a chemiluminescent reaction. The resulting chemiluminescent signal is measured at 612 nm and is an inverse function of the concentration of sirolimus in the sample. The amount of this signal is related to the presence and or amount of sirolimus in the sample.

In one example in accordance with the present disclosure, the assay format is ACMIA (Affinity Chromium dioxide Mediated Immuno Assay). For the ACMIA assay format, a reagent is employed that is a conjugate of sirolimus and chrome particles, which reagent may be designated as a first component. A second component is an antibody for sirolimus. This antibody, crosslinked to a reporter enzyme (for example, beta-galactosidase), is added to a reaction vessel in an excess amount, i.e., an amount greater than that required to bind all of the analyte that might be present in a sample. The antibody-enzyme conjugate is mixed with a sample suspected of containing sirolimus to allow the sirolimus analyte to bind to the antibody. Next, the chrome particle reagent is added to bind any excess antibody-enzyme conjugate. Then, a magnet is applied, which pulls all of the chrome particles and excess antibody-enzyme out of the suspension, and the supernatant is transferred to a final reaction container. The substrate of the reporter enzyme is added to the final reaction container, and the enzyme activity is measured spectrophotometrically as a change in absorbance over time. The amount of this signal is related to one or both of the presence and amount of sirolimus in the sample.

Another example of an assay format is an EMIT® assay. In this assay format, a conjugate of an enzyme and sirolimus is utilized where a medium comprising the conjugate comprises a chelating agent in accordance with the principles described herein. The enzyme may be G-6-PDH, by way of illustration and not limitation. An antibody for sirolimus is incubated with the enzyme-conjugate and a sample suspected of containing sirolimus. Antibody for sirolimus binds to the sirolimus analyte in the sample instead of binding to the enzyme conjugate, which reduces the amount of inhibition of the enzyme activity that might otherwise occur in the absence of sirolimus in the sample. In this way, samples with more sirolimus analyte will yield higher enzyme activity, and samples with no sirolimus analyte will have the maximum inhibition and the lowest enzyme activity. The amount of reduction of inhibition of enzyme activity is related to the amount of sirolimus in the sample.

Another particular example of an assay that may be employed for the determination of an immunosuppressant drug analyte is discussed in U.S. Pat. No. 5,616,719 (Davalian, et al.), which describes fluorescent oxygen channeling immunoassays and which disclosure is incorporated herein by reference.

### Kits

The reagents for conducting a particular assay may be present in a kit useful for conveniently performing an assay for the determination of an immunosuppressant drug analyte. In one example in accordance with the principles described herein, a kit comprises in packaged combination an antibody for an immunosuppressant drug analyte and other reagents for performing an assay, the nature of which depend upon the particular assay format. One reagent is a conjugate of an immunosuppressant drug and a conjugative moiety that also comprises a chelating agent in accordance with the principles described herein. The reagents may each be in separate containers or various reagents can be combined in one or more containers depending on the cross-reactivity and stability of the reagents. The kit can further include other separately packaged reagents for conducting an assay such as additional sbp members, sps members, and ancillary reagents such as an ancillary enzyme substrate, and so forth.

The relative amounts of the various reagents in the kits can be varied widely to provide for concentrations of the reagents that substantially optimize the reactions that need to occur during the assay method and further to optimize substantially the sensitivity of the assay. Under appropriate circumstances one or more of the reagents in the kit can be provided as a dry powder, usually lyophilized, including excipients, which on dissolution will provide for a reagent solution having the appropriate concentrations for performing a method or assay in accordance with the present invention. The kit can further include a written description of a method in accordance with the present invention as described above.

### Definitions

The phrase “at least” as used herein means that the number of specified items may be equal to or greater than the number recited.

The phrase “about” as used herein means that the number recited may differ by plus or minus 10%; for example, “about 5” means a range of 4.5 to 5.5.

### EXAMPLES

The following examples are by way of illustration and not limitation on the scope of the present disclosure and the appended claims. Numerous modifications and alternative compositions, methods, and systems may be devised without departing from the spirit and scope of the present disclosure. Unless otherwise indicated, materials in the experiments below may be purchased from Aldrich Chemical Company, St. Louis Mo. Parts and percentages are by weight unless indicated otherwise.

### Materials

Sirolimus reagent cartridges are obtained from Siemens Americas Distribution Center, Elkhart, Ind. In wells 1 and 2 the reagent cartridges contain 1800 μL of a reagent that is a conjugate of anti-sirolimus antibody and β-galactosidase; in wells 3 and 4, the reagent cartridges contain four biotinylated sirolimus::streptavidin dexal chromium dioxide tablets per well; in wells 5 and 6, the reagent cartridges contain two chlorophenol red-β-D-galactopyranoside (CPRG) tablets per well; in well 7, the reagent cartridges contain 5,000 μL of CPRG diluent (0.178 M HEPES buffer, pH 7.8, 12% ethylene glycol, 0.08M magnesium acetate, 0.1% PROCLIN® 300 biocide preservative) for CPRG tablet hydration; and in well 8, the reagent cartridges contain 2,800 μL of hemolytic pretreatment solution (see below).

Preparation of hemolytic pretreatment solution. This pretreatment solution is prepared to contain 15 μg/mL of a FK-506 carbamate compound (FKE), 6.8 mg/mL PIPES™ 1.5 sodium salt, 0.3 mg/mL ethylenediaminetetraacetate (EDTA) disodium, 1.0 mg/mL saponin, 0.2% PROCLIN® 300 biocide preservative, 0.024 mg/mL neomycin sulfate and 0.99 mg/mL NaN₃, pH 6.5. The FKE concentration in the final reaction mixture is 3.4 μg/mL.

Preparation of conjugate of anti-sirolimus antibody and β-galactosidase. Monoclonal anti-sirolimus antibody (Wyeth Pharmaceuticals, Cambridge, Mass.) is conjugated to β-galactosidase using a standard heterobifunctional SMCC (succinimidyl trans-4-(N-maleimidylmethyl)cyclohexane-1-carboxylate) linker. The antibody conjugate solution contains approximately 7.5 μg/mL of the conjugate of anti-sirolimus antibody and β-galactosidase, 30 mg/mL protease free bovine serum albumin, 0.126 mg/mL MgCl₂, 0.03 mL/mL of ethylene glycol, 35.14 mg/mL PIPES 1.5 sodium salt, 50 mg/mL NaCl and beta-gal mutein (inactivated beta-galactosidase), pH 6.5.

Magnetic chrome tablet preparation. Biotinylated sirolimus::streptavidin dexal chromium dioxide tablets are prepared with a molar ratio of 2 (biotin):1 (streptavidin). The streptavidin-coated chromium dioxide particles and biotinylated sirolimus are made as described in U.S. Pat. No. 7,842,475, the relevant portions of which are incorporated herein by reference. The reagents are incubated in a ratio as described above for 48 to 64 hrs to produce a chrome slurry, which is washed 10 times with 100 mM sodium phosphate buffer, pH 7.0, and then washed 5 times in 50 mM PIPES buffer, pH 6.5. The washed chrome slurry is held for 8 hours or until the supernatant becomes clear. The supernatant is then carefully removed to harvest the chrome slurry. A solution containing 8% CARBOWAX™ (polyethylene glycol 8000) and 84% trehalose is slowly added to the chrome slurry, which is mixed using a propeller mixer. The solution is mixed for 10 minutes before loading on a spray assembly and spray frozen using liquid nitrogen (LN₂) at an output temperature within a range of minus 140° C. to minus 75° C. Next, the resultant blend (frozen droplets or granules) is transferred and loaded immediately into a pre-cooled Hull Freeze dryer (SP Industries, Inc, Warminster, Pa.), where it is lyophilized for 5 days at less than minus 35° C. until it becomes a dry blend powder. The dried blend powder is collected and pressed to form 30 mg chrome tablets using a single station tablet press (Advanced Machinery, MI 48035).

On a Dimension RxL/HM instrument (Siemens Healthcare Diagnostics Inc., Deerfield, Ill.), four chromium dioxide tablets are hydrated with 1900 mL of deionized water with ultrasound mixing to re-suspend the particles. For chrome tablets with added EDTA, 1.8 mg of N,N′-1,2-ethanediylbis [N-(carboxymethyl)- glycine], disodium salt, dehydrate is added per tablet. Table 1 shows the formulation of the tablets with added EDTA and without EDTA.

TABLE 1

| | |
|---|---|
| Ingredient | mg/Tablet |

Sirolimus chrome formulations with added EDTA

| | |
|---|---|
| biotinylated sirolimus::streptavidin | 2.4 |
| dexal chromium dioxide | |
| Trehalose | 31 |
| Carbowax | 2.8 |
| EDTA (C₁₀H₁₄O₈N₂Na₂•2H₂O) | 1.768 |

Sirolimus chrome formulations without EDTA

| | |
|---|---|
| biotinylated sirolimus::streptavidin | 2.4 |
| dexal chromium dioxide | |
| Trehalose | 33 |
| Carbowax | 2.8 |

Sirolimus assay. The measurement of sirolimus is carried out using the assay format known as ACMIA. The principle and operation of an ACMIA sirolimus assay method are as follows: a 70 μL of pretreatment reagent containing surfactants and a FKE (U.S. Pat. No. 7,186,518, the relevant disclosure of which is incorporated herein by reference) is added to the reaction vessel on a DIMENSION® chemistry RxL instrument with HM Module (Siemens Healthcare Diagnostics Inc., Deerfield, Ill.). Next, 18 μL of whole blood containing sirolimus is added. The whole blood is sampled from a standard cup by first mixing the blood with the ultrasonic sample probe. The mixing of whole blood sample with the pretreatment solution containing surfactants and FKE ensures the lysis of the whole blood and the displacement of the protein bound sirolimus molecules from their binding sites by the FKE molecules. A conjugate of anti-sirolimus antibody and β-galactosidase (50 μL) is added and allowed to react with sirolimus in the sample. Four 35 mg dry CrO₂ tablets, each containing 2.5 mg of preformed chrome particles or rapamycin-DA10-dexal-chrome particles as described in U.S. Pat. No. 7,842,475, the relevant disclosure of which is incorporated herein by reference, 8% trehalose, and 84% CARBOWAXTM (polyethylene glycol 8000), are hydrated with 1900 μL of water on board the DIMENSION® instrument with an ultrasonic reagent probe. Then, 50 μL of the hydrated chrome particles is added to the reaction mixture and allowed to bind the unreacted conjugate. The sirolimus bound by the conjugate of anti-sirolimus antibody and β-galactosidase does not bind to the chrome particles but remains in the supernatant when a magnetic field is applied to the above reaction mixture to separate the solution from the chrome particles. The sirolimus bound conjugate is detected by transferring the supernatant from the reaction vessel to a photometric cuvette and measuring the enzymatic rate of the conjugate in the presence of chlorophenol red-β-D-galactopyranoside (CPRG). The rate is measured bichromatically at 577 and 700 nm.

Reagent stability of sirolimus-CrO₂ tablet spiked with EDTA solution. The sirolimus assay is conducted as described above using the CrO₂ tablets spiked with EDTA disodium and without spiked EDTA. The stability of the CrO₂ reagents is then monitored for 2 days post hydration by testing a quality control material (Rapamycin (Rap)/Tacrolimus (Tac)/Cyclosporin A (CsA) control level 1) from More Diagnostics (Los Osos, Calif.). The mean target value of the quality control material is 2.6±0.39 ng/mL of sirolimus. Table 2 summarizes the data, which demonstrate the improved hydrated stability of the sirolimus-CrO₂ spiked with EDTA as compared to sirolimus-CrO₂ without spiked EDTA.

TABLE 2
Stability Comparison Between Siro-CrO₂ Spiked with
EDTA and without EDTA

| Days after | CrO₂ + EDTA | | CrO₂ − EDTA | |
|---|---|---|---|---|
| Hydration | Mean ng/mL | SD ng/mL | Mean ng/mL | SD ng/mL |
| 0 | 2.2 | 0.31 | 2.2 | 0.18 |
| 1 | 2.4 | 0.25 | 2.6 | 0.30 |
| 2 | 2.4 | 0.28 | 3.3 | 0.95 |

Reagent stability of sirolimus-CrO₂ tablet containing EDTA. The sirolimus assay is conducted as described above using the CrO₂ tablets made with and without EDTA. The stability of the CrO₂ reagents is then monitored for 2 days post hydration by testing a quality control material (Rap/Tac/CsA control level 1) from More Diagnostics. The mean target value of the quality control material is 2.6±0.39 ng/mL of sirolimus.

Table 3 summarizes the data, which demonstrate the improved hydrated stability of the sirolimus-CrO₂ with EDTA as compared to sirolimus-CrO₂ without EDTA.

TABLE 3
Stability Comparison Between Sirolimus-CrO2 with
and without EDTA

| Days after | CrO₂ + EDTA | | CrO₂ − EDTA | |
|---|---|---|---|---|
| Hydration | Mean ng/mL | SD ng/mL | Mean ng/mL | SD ng/mL |
| 0 | 2.3 | 0.15 | 2.3 | 0.17 |
| 1 | 2.2 | 0.17 | 2.4 | 0.20 |
| 2 | 2.2 | 0.20 | 2.9 | 0.23 |

Although the foregoing invention has been described in some detail by way of illustration and example for purposes of clarity of understanding, it will be readily apparent to those of ordinary skill in the art in light of the teachings in accordance with the principles described herein that certain changes and modifications may be made thereto without departing from the spirit or scope of the appended claims. Furthermore, the foregoing description, for purposes of explanation, used specific nomenclature to provide a thorough understanding of the examples. It will be apparent to one skilled in the art that the specific details are not required in order to practice the examples described herein. Thus, the foregoing descriptions of specific examples in accordance with the principles described herein are presented for purposes of illustration and description; they are not intended to be exhaustive or to limit the examples to the precise forms disclosed. Many modifications and variations are possible in view of the above teachings. The examples were chosen in order to explain the principles described herein and their practical applications and to thereby enable others skilled in the art to utilize the teachings.

What is claimed is:

1. A method of maintaining the antibody-binding activity of a conjugate of an immunosuppressant drug and a conjugative moiety, said method comprising combining the conjugate and an effective amount of a chelating agent.

2. The method according to claim 1 wherein the immunosuppressant drug is sirolimus, everolimus, rapamycin, tacrolimus or cyclosporin.

3. The method according to claim 1 wherein the conjugative moiety is selected from the group consisting of immunogenic carriers, particles and labels.

4. The method according to claim 1 wherein the chelating agent is selected from the group consisting of ethylene diamine tetraacetate and its salts and ethylene glycol tetraacetic acid and its salts.

5. A method of maintaining the antibody-binding activity of a conjugate of sirolimus and a conjugative moiety, said method comprising combining with said conjugate an effective amount of a chelating agent.

6. The method according to claim 5 wherein the conjugative moiety is selected from the group consisting of immunogenic carriers, particles and labels.

7. The method according to claim 1 wherein the chelating agent is selected from the group consisting of ethylene diamine tetraacetate and its salts and ethylene glycol tetraacetic acid and its salts.

8. A composition comprising in an aqueous medium (i) a conjugate of an immunosuppressant drug and a conjugative moiety and (ii) a chelating agent in an amount effective to maintain an antibody-binding activity of the conjugate of the immunosuppressant drug and the conjugative moiety.

9. The composition according to claim 8 wherein the immunosuppressant drug is sirolimus, everolimus, rapamycin, or cyclosporin.

10. The composition according to claim 8 wherein the conjugative moiety is selected from the group consisting of immunogenic carriers, particles and labels.

11. The composition according to claim 8 wherein the chelating agent is selected from the group consisting of salts of ethylene diamine tetraacetic acid and ethylene glycol tetraacetic acid and its salts.

12. A method for detecting an immunosuppressant drug in a sample, said method comprising:
(a) providing in combination in an assay medium a sample suspected of containing an immunosuppressant drug, an antibody for the immunosuppressant drug and a composition according to claim 8 wherein the conjugative moiety is a particle,
(b) examining the combination for the formation of a complex between the antibody for the immunosuppressant drug and the immunosuppressant drug of the sample or for the formation of a complex between the antibody for the immunosuppressant drug and the immunosuppressant drug of the conjugate, and
(c) relating the presence of the complex to one or both of a presence and an amount of the immunosuppressant drug in the sample.

13. The method according to claim 12 wherein the combination further comprises a second antibody wherein the second antibody comprises a label and binds to the complex.

14. The method according to claim 12 wherein the antibody for the immunosuppressant drug comprises a label.

15. A method for detecting an immunosuppressant drug in a sample, said method comprising:
(a) providing in combination in an assay medium a sample suspected of containing an immunosuppressant drug, an antibody for the immunosuppressant drug and a composition according to claim 8 wherein the conjugative moiety is a label,
(b) examining the combination for the formation of a complex between the antibody for the immunosuppressant drug and the immunosuppressant drug, and
(c) relating the presence of the complex to one or both of a presence and an amount of the immunosuppressant drug in the sample.

16. A composition comprising in an aqueous medium (i) a conjugate of sirolimus and a conjugative moiety and (ii) an ethylene diamine tetraacetic acid reagent in an amount effective to maintain an antibody-binding activity of the conjugate of the sirolimus and the conjugative moiety.

17. The composition according to claim 16 wherein the conjugative moiety is selected from the group consisting of immunogenic carriers, particles and labels.

18. A method for detecting sirolimus in a sample, said method comprising:
(a) providing in combination in an assay medium a sample suspected of containing sirolimus, an antibody for sirolimus and a composition according to claim 16 wherein the conjugative moiety is a particle,
(b) examining the combination for the formation of a complex between the antibody for sirolimus and the sirolimus of the sample or for the formation of a complex between the antibody for sirolimus and the sirolimus of the conjugate, and
(c) relating the presence of the complex to one or both of a presence and an amount of sirolimus in the sample.

19. The method according to claim 18 wherein the combination further comprises a second antibody wherein the second antibody comprises a label and binds to the complex.

20. The method according to claim 18 wherein the antibody for sirolimus comprises a label.

21. A method for detecting sirolimus in a sample, said method comprising:
(a) providing in combination in an assay medium a sample suspected of containing sirolimus, an antibody for sirolimus and a composition according to claim 16 wherein the conjugative moiety is a label,
(b) examining the combination for the formation of a complex between the antibody for sirolimus and the sirolimus of the sample, and
(c) relating the presence of the complex to one or both of a presence and an amount of sirolimus in the sample.
